(19) United States
(12) Patent Application Publication
(10) Pub. No.: US 2026/0340827 A1
(43) Pub. Date: Oct. 29, 2026

(54) METHOD FOR ANALYZING COMPOSITION OF URINARY CALCULUS

(71) Applicant: The First Affiliated Hospital of Guangzhou Medical University (Guangzhou Respiratory Center), Guangzhou (CN)

(72) Inventors: Xinchun LI, Guangzhou (CN); Lisheng CHEN, Guangzhou (CN); Xian LI, Guangzhou (CN); Yingjian YANG, Shenzhen (CN); Tianqi WU, Shenzhen (CN); Jie ZHENG, Shenzhen (CN); Peng GUO, Shenzhen (CN)

(73) Assignee: The First Affiliated Hospital of Guangzhou Medical University (Guangzhou Respiratory Center), Guangzhou (CN)

(21) Appl. No.: 19/653,181

(22) Filed: Apr. 21, 2026

(30) Foreign Application Priority Data
Apr. 27, 2025 (CN) 202510543082.2

(51) Int. Cl. G06T 7/00

(52) U.S. Cl. CPC G06T 7/0014; G06T 2207/30084

(57) ABSTRACT

The technique can be used for urinary calculus type identification, comprising: using an atomic number image of a urinary calculus corresponding to a dual-energy abdominal image; extracting a plurality of composition percentages of corresponding peaks of a plurality of histograms corresponding to effective atomic numbers in the atomic number image of the urinary calculus; and identifying, on the basis of a plurality of composition percentages and a plurality of pre-set calculus type composition percentage ranges, a calculus type, so as to meet the clinical diagnostic requirement that mixed-composition calculi are more frequent than single-composition calculi.

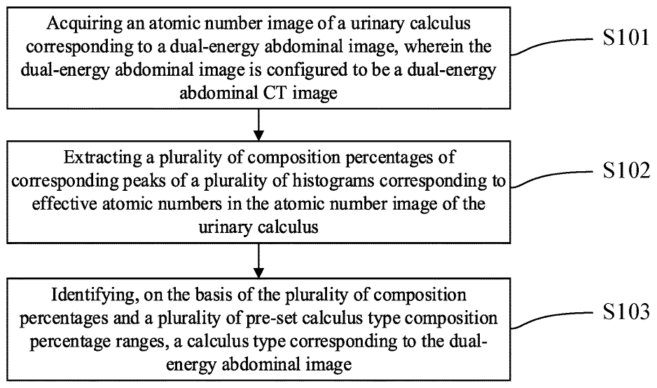

### CROSS-REFERENCE TO RELATED APPLICATIONS

The application claims priority to Chinese patent application No. 202510543082.2, filed on Apr. 27, 2025, the entire contents of which are incorporated herein by reference.

### TECHNICAL FIELD

The present disclosure relates to the technical field of urinary calculus composition analysis, and in particular to a method for analyzing the composition of a urinary calculus.

### BACKGROUND

The urinary calculus is a common disease, among which renal calculi are the most frequent. The urinary calculus can cause clinical symptoms such as pain, dysuria, infection, hematuria, and the like. Without timely treatment, they may easily lead to renal function damage or even renal failure, and may also increase the incidence of many chronic diseases, including osteoporosis and cardiovascular diseases. Identifying the composition of a urinary calculus is of great significance for selecting appropriate treatment methods according to different compositions and intervention and guidance to prevent recurrence. For example, urinary alkalinization dissolution may be the first choice for a uric acid calculus, and surgical treatment may be performed if necessary; a struvite/magnesium ammonium phosphate calculus usually requires preoperative antibiotic therapy; and a calcium oxalate calculus, a carbonate apatite calculus and a calcium carbonate phosphate calculus often require surgical treatment. In order to prevent the recurrence of a urinary calculus, it is often necessary to restrict and modify a patient's diet according to the calculus composition, identify relevant metabolic diseases, and administer pharmaceutical treatment.

### SUMMARY

According to the embodiments of the present disclosure, a method for analyzing the composition of a urinary calculus is provided, applied to calculus type identification, comprising:
acquiring an atomic number image of a urinary calculus corresponding to a dual-energy abdominal image;
extracting a plurality of composition percentages of corresponding peaks of a plurality of histograms corresponding to effective atomic numbers in the atomic number image of the urinary calculus;
identifying, on the basis of the plurality of composition percentages and a plurality of pre-set calculus type composition percentage ranges, a calculus type corresponding to the dual-energy abdominal image, comprising: if at least two of the plurality of composition percentages fall within the composition percentage ranges, then acquiring an iodine-based image corresponding to the dual-energy abdominal image; determining a calculus gray-scale value of the iodine-based image;
respectively calculating an average gray-scale value and a uniformity corresponding to the calculus gray-scale value; if the average gray-scale value is greater than a set average gray-scale value and the uniformity is greater than a set uniformity, then configuring the calculus type to be calcium oxalate dihydrate calculus; otherwise, configuring the calculus type to be struvite calculus or carbonate apatite calculus.

### BRIEF DESCRIPTION OF DRAWINGS

The accompanying drawings herein illustrate embodiments consistent with the present disclosure and, together with the description, serve to explain the technical solutions of the present disclosure.

The figure shows a flow chart of a method for analyzing the composition of a urinary calculus according to an embodiment of the present disclosure.

### DETAILED DESCRIPTION OF THE EMBODIMENTS

Various exemplary embodiments, features, and aspects of the present disclosure will be described in detail below with reference to the accompanying drawings. The word “exemplary” is used herein to mean “as an example, instance, or illustration.” Any embodiment described herein as “exemplary” is not necessarily to be construed as preferred or advantageous over other embodiments.

In addition, numerous specific details are provided in the following detailed description in order to better describe the present disclosure. Those skilled in the art should appreciate that the present disclosure may be practiced without some of these specific details.

The figure shows a flow chart of a method for analyzing the composition of a urinary calculus according to an embodiment of the present disclosure. As shown in the figure, the method for analyzing the composition of a urinary calculus is applied to calculus type identification, comprising: step S101: acquiring an atomic number image of a urinary calculus corresponding to a dual-energy abdominal image, wherein the dual-energy abdominal image is configured to be a dual-energy abdominal CT image; step S102: extracting a plurality of composition percentages of corresponding peaks of a plurality of histograms corresponding to effective atomic numbers in the atomic number image of the urinary calculus; and step S103: identifying, on the basis of the plurality of composition percentages and a plurality of pre-set calculus type composition percentage ranges, a calculus type corresponding to the dual-energy abdominal image. This is to solve the technical problem that it is difficult to distinguish or identify mixed urinary calculi of different compositions, thereby failing to meet the clinical diagnostic requirement that mixed-composition calculi are more frequent than single-composition calculi.

In one embodiment, the abdominal region of a patient is scanned by using a spectral CT equipment (a dual-energy CT equipment or a spectral CT equipment) to obtain a three-dimensional dual-energy abdominal image (a dual-energy spectral abdominal image, a dual-energy abdominal CT image, a dual-energy spectral abdominal CT image, a spectral abdominal image or a spectral abdominal CT image).

In one embodiment, the spectral CT equipment is a rapid tube voltage switch dual-source CT equipment, adopts an instantaneous kVp switching technique to complete high and low monoenergetic switching within an extremely short time (<0.25 ms), realizing same time, same orientation, and same source of dual-energy. It overcomes the defects of dual-tube dual-source CT, thereby avoiding a slight angular difference between scanning planes of two tubes, and improving the accuracy of acquiring data so as to provide a superior CT scanning image.

In one embodiment, spectral CT scanning and three-dimensional reconstruction can not only clearly demonstrate the density and morphology of renal parenchyma, the orientation and contour of renal pelvis and calyces, the course and lumen of ureter, as well as the bladder wall and intraluminal conditions, but also show the morphology and positions of a renal calculus and the renal pelvis and calyces, and further measure the volumes of the calculus and the renal pelvis and calyces. The imaging quality is far superior to that of three-dimensional multi-planar reconstruction of conventional CT. The effective atomic number (Zeff value) of spectral CT can be used to analyze the composition characteristics of the renal show numerical differences in different regions, and clarify the specific spatial distribution of different compositions in a mixed calculus based on these differences.

Step S102: extracting a plurality of composition percentages of corresponding peaks of a plurality of histograms corresponding to effective atomic numbers in the atomic number image of the urinary calculus.

In one embodiment, a method for extracting the plurality of composition percentages comprises: extracting a plurality of histograms corresponding to effective atomic numbers in the atomic number image of the urinary calculus; and determining, according to the plurality of histograms corresponding to the atomic number image of the urinary calculus, a plurality of composition percentages of corresponding peaks of the plurality of histograms.

In one embodiment, step S102 further comprises: acquiring a set histogram color and/or a set histogram width; extracting, according to the set histogram color and/or set histogram width, a plurality of histograms corresponding to effective atomic numbers, which satisfy the set histogram color and/or set histogram width, in the atomic number image of the urinary calculus; and determining, according to the plurality of corresponding histograms, which satisfy the set histogram color and/or set histogram width, in the atomic number image of the urinary calculus, a plurality of composition percentages of corresponding peaks of the plurality of histograms.

In one embodiment, those skilled in the art may configure the set histogram color and/or set histogram width according to actual needs. For example, the set histogram color is configured to be yellow.

In one embodiment, before said extracting a plurality of histograms corresponding to effective atomic numbers in the atomic number image of the urinary calculus, or said extracting a plurality of histograms corresponding to effective atomic numbers, which satisfy the set histogram color and/or set histogram width, in the atomic number image of the urinary calculus, the atomic number image of the urinary calculus is corrected to obtain a corrected atomic number image of the urinary calculus; and a plurality of composition percentages of corresponding peaks of a plurality of histograms corresponding to effective atomic numbers in the corrected atomic number image of the urinary calculus are extracted, or a plurality of composition percentages of corresponding peaks of a plurality of histograms corresponding to effective atomic numbers which satisfy the set histogram color and/or set histogram width in the corrected atomic number image of the urinary calculus are extracted.

In one embodiment, step S102 further comprises: performing at least one correction processing operation such as angle adjustment, contrast enhancement, and scaling on the atomic number image of the urinary calculus to obtain a corrected atomic number image of the urinary calculus, so as to facilitate subsequent extraction of a plurality of histograms corresponding to the effective atomic numbers.

In one embodiment, an optical character recognition technique is used to extract a plurality of composition percentages of corresponding peaks of a plurality of histograms corresponding to effective atomic numbers in the atomic number image of the urinary calculus; or the optical character recognition technique is used to extract a plurality of composition percentages of corresponding peaks of a plurality of histograms corresponding to effective atomic numbers which satisfy the set histogram color and/or set histogram width in the corrected atomic number image of the urinary calculus.

In one embodiment, optical character recognition (OCR) is a computer vision technique that recognizes and extracts text content from an image through image processing and machine learning algorithms, and converts same into a machine-readable and editable text format.

In an embodiment of the present disclosure, step S102 further comprises: acquiring a set composition percentage that is smaller than the range minimum value in the set calculus type composition percentage range; if the composition percentages corresponding to a plurality of histograms are smaller than the set composition percentage, then deleting the composition percentage which is smaller than the set composition percentage from the plurality of composition percentages corresponding to the plurality of histograms; otherwise, retaining the composition percentages which are greater than or equal to the set composition percentage; and identifying, on the basis of the retained plurality of composition percentages and a plurality of set calculus type composition percentage ranges, the calculus type corresponding to the dual-energy abdominal image.

In one embodiment, those skilled in the art may configure the set composition percentage that is smaller than the range minimum value in the set calculus type composition percentage range according to actual needs. For example, the set composition percentage that is smaller than the range minimum value in the set calculus type composition percentage range is configured to be 5%.

In one embodiment, the implementation of OCR requires calling the API of the GOT_OCR2 tool and setting its parameters such as language and text box positioning; and the identification of atomic number peaks and morphological calculation require using the NumPy, SciPy, or OpenCV library of Python to extract a plurality of composition percentages of corresponding peaks of a plurality of histograms corresponding to effective atomic numbers in the atomic number image of the urinary calculus, or a plurality of composition percentages of corresponding peaks of a plurality of histograms corresponding to effective atomic numbers which satisfy the set histogram color and/or set histogram width in the atomic number image of the urinary calculus.

Step S103: identifying, on the basis of the plurality of composition percentages and a plurality of pre-set calculus type composition percentage ranges, a calculus type corresponding to the dual-energy abdominal image.

In one embodiment, step S103 further comprises: if a composition percentage among the plurality of composition percentages falls within a certain set calculus type composition percentage range among the plurality of set calculus type composition percentage ranges, configuring the calculus type corresponding to the certain set calculus type composition percentage range to be a calculus type corresponding to the composition percentage within the range.

In one embodiment, if at least two of the plurality of composition percentages fall within the corresponding set calculus type composition percentage range, then an iodine-based image corresponding to the dual-energy abdominal image is acquired; and on the basis of the iodine-based image, the calculus type corresponding to the overlapping range is identified.

In one embodiment, step S103 further comprises: determining a calculus gray-scale value of the iodine-based image; respectively calculating an average gray-scale value and a uniformity corresponding to the calculus gray-scale value; if the average gray-scale value is greater than a set average gray-scale value and the uniformity is greater than a set uniformity, then configuring the calculus type to be calcium oxalate dihydrate calculus; otherwise, configuring the calculus type to be struvite calculus or carbonate apatite calculus.

In one embodiment, those skilled in the art may configure the set average gray-scale value or the set uniformity according to actual needs.

In one embodiment, uniformity=(maximum value-minimum value)/(2*average value)×100%, where the maximum value represents the maximum value corresponding to the calculus gray-scale value, the minimum value represents the minimum value corresponding to the calculus gray-scale value, and the average value represents the average gray-scale value corresponding to the calculus gray-scale value.

In an embodiment of the present disclosure, the plurality of composition percentage ranges of set calculus types comprise: one or more of the following: a composition percentage range of uric acid calculus, a composition percentage range of calcium oxalate monohydrate calculus, a composition percentage range of calcium oxalate dihydrate calculus, a composition percentage range of carbonate apatite calculus, a composition percentage range of calcium carbonate phosphate calculus, and a composition percentage range of struvite calculus.

In one embodiment, the composition percentage range of uric acid calculus is configured to be 6.5-10.5.

In one embodiment, the composition percentage range of calcium oxalate monohydrate calculus is configured to be 13.3-14.0.

In one embodiment, the composition percentage range of calcium oxalate dihydrate calculus is configured to be 12.0-13.3.

In one embodiment, the composition percentage range of carbonate apatite calculus is configured to be 14.0-15.0.

In one embodiment, the composition percentage range of calcium carbonate phosphate calculus is configured to be greater than 12.5.

In one embodiment, the composition percentage range of struvite calculus is configured to be smaller than 12.5.

In one embodiment, the Zeff peak (composition percentage range) of uric acid calculus is 6.5-10.5.

In one embodiment, the Zeff peak of calcium oxalate monohydrate calculus is 13.3-14.0.

In one embodiment, the Zeff peak of calcium oxalate dihydrate calculus is 12.0-13.3.

In one embodiment, the Zeff peak of carbonate apatite calculus is 14.0-15.0.

In one embodiment, the Zeff peak of calcium carbonate phosphate calculus is greater than 12.5, and the CT value images all show inhomogeneous density.

In one embodiment, the Zeff peak of struvite calculus is smaller than 12.5, and the CT value images all show inhomogeneous density.

In one embodiment, the method for identifying, on the basis of the plurality of composition percentages and a plurality a plurality of pre-set calculus type composition percentage ranges, a calculus type corresponding to the dual-energy abdominal image, comprises: constructing a calculus composition comparison table according to the plurality of set composition percentage ranges of calculus types; and performing, on the basis of the calculus composition comparison table, a table lookup and comparison operation on the plurality of composition percentages, so as to identify the calculus type corresponding to the dual-energy abdominal image.

Various embodiments of the present disclosure have been described above. The description above is exemplary, not exhaustive, and is not limited to the disclosed embodiments. Many modifications and variations will be apparent to those of ordinary skill in the art without departing from the scope and spirit of the described embodiments. The terminology used herein is chosen to best explain the principles of the embodiments, their practical application, or improvements over the technologies in the market, or to enable other persons of ordinary skill in the art to understand the embodiments disclosed herein.

What is claimed is:

1. A method for analyzing a composition of a urinary calculus, applied to calculus type identification, comprising:
acquiring an atomic number image of a urinary calculus corresponding to a dual-energy abdominal image;
extracting a plurality of composition percentages of corresponding peaks of a plurality of histograms corresponding to effective atomic numbers in the atomic number image of the urinary calculus;
identifying, on the basis of the plurality of composition percentages and a plurality of pre-set calculus type composition percentage ranges, a calculus type corresponding to the dual-energy abdominal image, comprising: if at least two of the plurality of composition percentages fall within the composition percentage ranges, then acquiring an iodine-based image corresponding to the dual-energy abdominal image; determining a calculus gray-scale value of the iodine-based image;
respectively calculating an average gray-scale value and a uniformity corresponding to the calculus gray-scale value; if the average gray-scale value is greater than a set average gray-scale value and the uniformity is greater than a set uniformity, then configuring the calculus type to be calcium oxalate dihydrate calculus; otherwise, configuring the calculus type to be struvite calculus or carbonate apatite calculus.

2. The method of claim 1, wherein extracting the plurality of composition percentages comprises:
acquiring a set histogram color or a set histogram width;
extracting, according to the set histogram color or the set histogram width, a plurality of histograms corresponding to effective atomic numbers, which satisfy the set histogram color or the set histogram width, in the atomic number image of the urinary calculus; and
determining, according to the plurality of histograms, a plurality of composition percentages of corresponding peaks of the plurality of histograms.

3. The method of claim 1, further comprising:
acquiring a set composition percentage that is smaller than a range minimum value in the set calculus type composition percentage range;
if the composition percentages corresponding to a plurality of histograms are smaller than the set composition percentage, then deleting the composition percentage which is smaller than the set composition percentage from the plurality of composition percentages corresponding to the plurality of histograms;
otherwise, retaining the composition percentages which are greater than or equal to the set composition percentage; and
identifying, on the basis of the retained plurality of composition percentages and a plurality of set calculus type composition percentage ranges, the calculus type corresponding to the dual-energy abdominal image.

4. The method of claim 3, further comprising:
if a composition percentage among the plurality of composition percentages falls within a certain set calculus type composition percentage range among the plurality of set calculus type composition percentage ranges,
configuring the calculus type corresponding to the certain set calculus type composition percentage range to be a calculus type corresponding to the composition percentage within the range.

5. The method of claim 1, wherein the composition percentage ranges comprise one or more of:
a composition percentage range of uric acid calculus, a composition percentage range of calcium oxalate monohydrate calculus, a composition percentage range of calcium oxalate dihydrate calculus, a composition percentage range of carbonate apatite calculus, a composition percentage range of calcium carbonate phosphate calculus, and a composition percentage range of struvite calculus.

6. The method of claim 5, wherein the composition percentage range of uric acid calculus is configured to be 6.5-10.5.

7. The method of claim 5, wherein the composition percentage range of calcium oxalate monohydrate calculus is configured to be 13.3-14.0.

8. The method of claim 5, wherein the composition percentage range of calcium oxalate dihydrate calculus is configured to be 12.0-13.3.

9. The method of claim 5, wherein the composition percentage range of carbonate apatite calculus is configured to be 14.0-15.0.

10. The method of claim 5, wherein the composition percentage range of calcium carbonate phosphate calculus is configured to be greater than 12.5.

11. The method of claim 5, wherein the composition percentage range of struvite calculus is configured to be smaller than 12.5.
